(19) United States
(12) Patent Application Publication
(10) Pub. No.: US 2025/0166805 A1
(43) Pub. Date: May 22, 2025

(54) FUNCTIONAL RESTRICTION OF A SYSTEM BASED ON INFORMATION FROM ANOTHER INDEPENDENT SYSTEM

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Kevin M. Fiebig, Cincinnati, OH (US); Charles J. Scheib, Loveland, OH (US); Luis Andrade Baez, JR., La Honda, CA (US)

(21) Appl. No.: 18/810,283

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. G16H 40/40

(52) U.S. Cl. CPC G16H 40/40

(57) ABSTRACT

Devices, systems, and techniques for functional restriction of a device based on information from another independent system. An example first surgical device may determine that the first surgical device is capable of obtaining medical information from a second surgical device. The first surgical device may receive the medical information from the second surgical device. The first surgical device may determine that the medical information satisfies a condition. Determining that the medical information satisfies a condition may involve determining that operation of the first surgical device and operation of the second surgical device have created a negative feedback loop that negatively impacts patient health. The first surgical device may limit functional capabilities of the first surgical device based on the medical information satisfying the condition.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

During an operation, multiple devices may have an effect on a patient. The devices may impact the functionality of other devices as a result. For example, two separate devices may contribute to a negative feedback loop that lowers the patient's core temperature indefinitely, which will harm the patient if left unchecked. The devices may have no knowledge of each other or the effects each has on the other or the patient. The devices may therefore be incapable of correcting the negative feedback loop.

A first system may apply conditional restrictions on its function or operation based on the function or operation of a second system. Conditional bounding of a first system may be based on the monitoring from the second system. For example, a first system may determine to not use its full operational capabilities based on information from a second system that relates to the first system's behavior.

The first system may include a control system for monitoring and controlling the operation of the first system, and the second system may include an independent control system for monitoring and controlling the operation of the second system. The second system may monitor at least one parameter that is relevant to the operation of the first system. The first system may not be monitoring the parameter(s). The second system may communicate with the first system to provide the first system with access to the data collected by the second system. The first system may use the information to alter operational bounding of the first system operation.

The systems may use directional synchronization to control the effect of the systems' operations on a physiologic parameter of the patient (e.g., when the physiological parameter is out of pre-established bounds). In some examples, predefined upper and/or lower bounds may not be used. Instead, the systems may use the outcome of the system operations as a metric of whether to limit the operations. For example, if the system operations are adjusted and result in better performance/impact on the patient, allow the adjustment. Similarly, if the system operations result in undesired behavior/impact, limit the operation to reduce the undesired consequences.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example surgical operating room with surgical instruments that may create a negative feedback loop.

FIG. 6 is a block diagram illustrating example components in surgical devices.

FIG. 7 illustrates an example feedback loop between two surgical devices.

FIG. 8A illustrates an example feedback loop between a smoke evacuator and a CO₂ insufflator.

FIG. 8B is a graph illustrating the feedback loop between the smoke evacuator and the CO₂ insufflator.

FIG. 9A illustrates an example feedback loop between a systemic warming device and a local cooling system.

FIG. 9B is a graph illustrating the feedback loop between the systemic warming device and the local cooling system.

FIG. 10 illustrates an example method that may be performed by a surgical device.

### DETAILED DESCRIPTION

A more detailed understanding may be had from the following description, given by way of example in conjunction with the accompanying drawings.

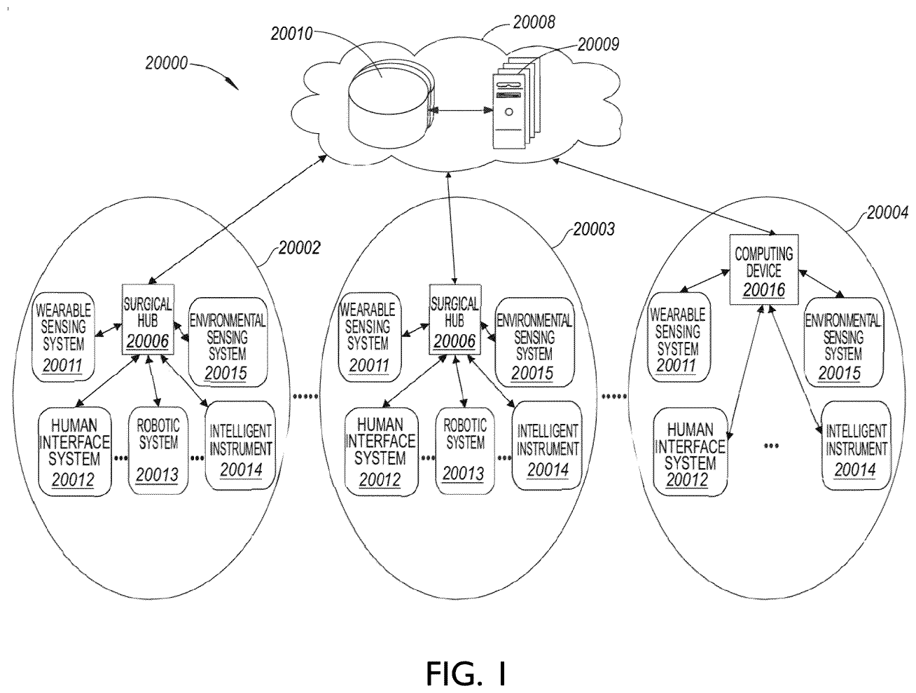

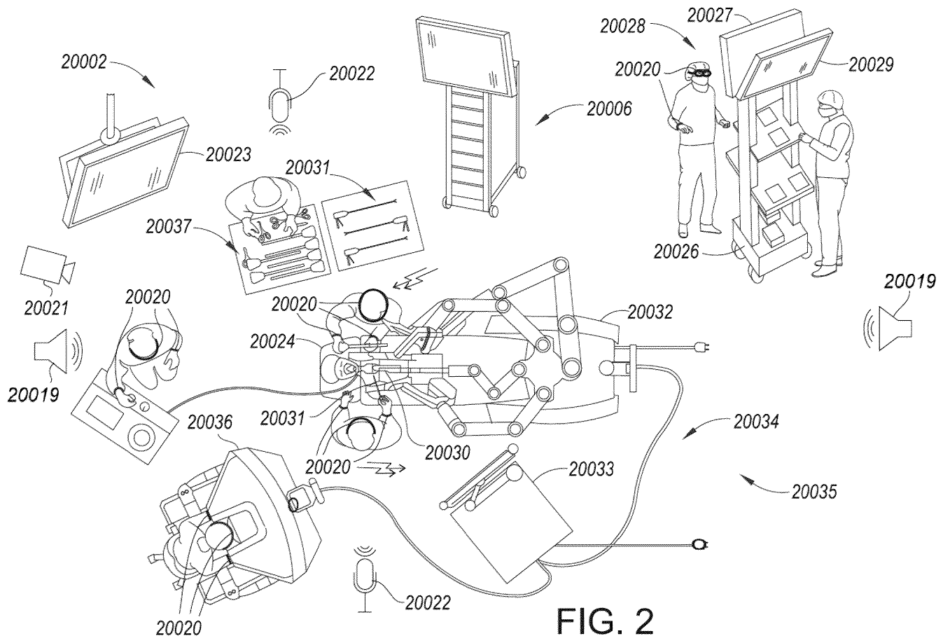

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

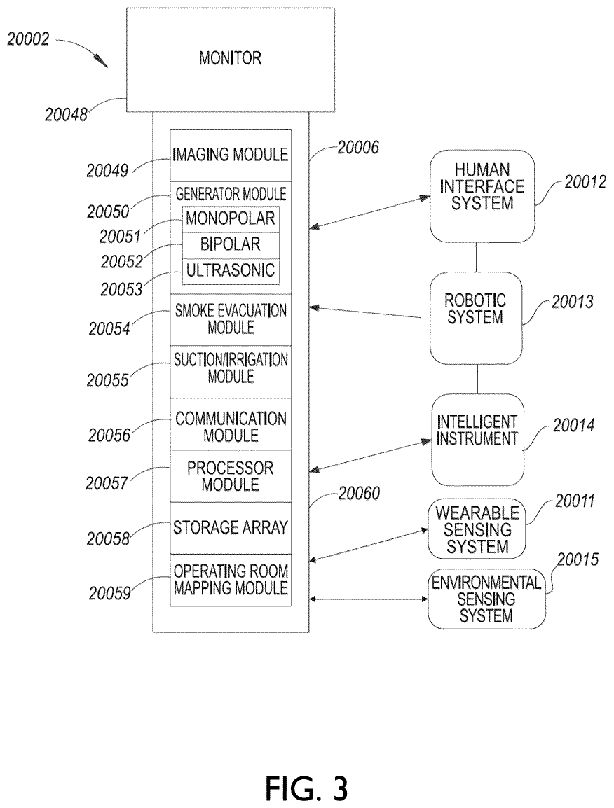

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

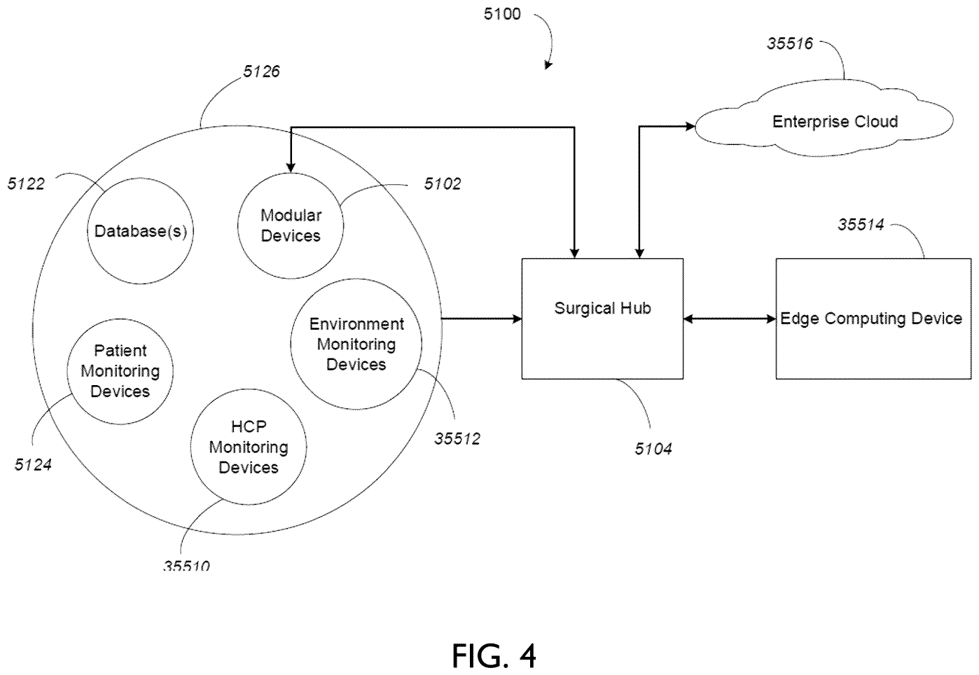

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

In some examples, systems may interact to create acceptable directional reinforcement. In this case, the systems may create a beneficial amplification based on directional adjustments. For example, an advanced energy monopolar device may increase its power level as the tissue desiccates, causing local heating of the electrode tip. A smart irrigation device may increase its flow rate to compensate for the additional heat at the electrode tip. The increase irrigation may increase the amount of saline in the region of the coagulation. The increased saline may increase the conductivity of the tissue. The increased conductivity may increase the effectiveness of the energy to further heat the tissue (e.g., both system are increasing the ability to apply heat which is the desired effect).

However, some systems may cause a cascading failure (e.g., self-reinforcing response). This is a failure in a system of interconnected parts in which the failure of one or few parts leads to the failure of other parts, growing progressively as a result of positive feedback.

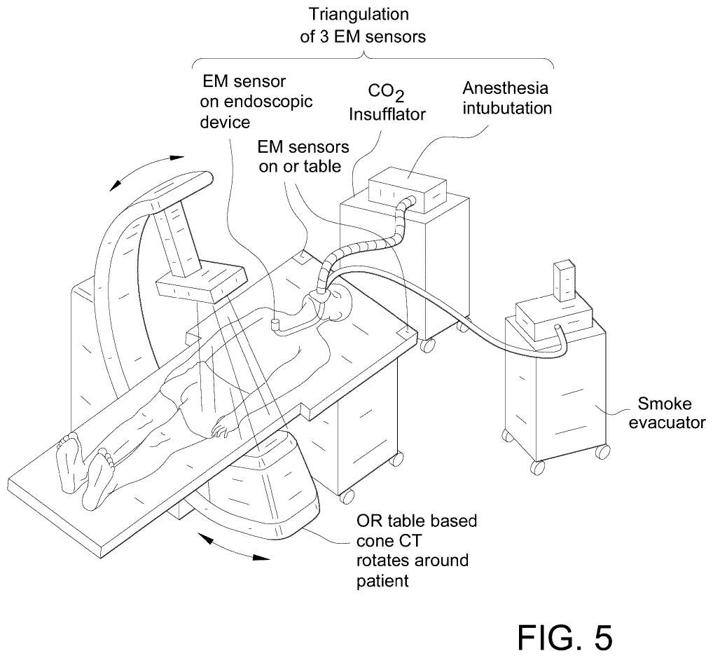

During an operation, multiple devices may have an effect on a patient. The devices may impact the functionality of other devices as a result. For example, two separate devices may contribute to a negative feedback loop that lowers the patient's core temperature indefinitely, which will harm the patient if left unchecked. The devices may have no knowledge of each other or the effects each has on the other or the patient. The devices may therefore be incapable of correcting the negative feedback loop. FIG. 5 illustrates an example surgical operating room in which two or more of the devices may create a negative feedback loop in the patient.

A first system may apply conditional restrictions on the function or operation of itself and/or a second system. For example, the first system may apply conditional bounding its functions based on monitoring from the second system. There may not be a (pre)defined upper or lower bounds. Instead, the systems may track the outcome of their actions, and use the outcome to determine a limit. For example, the systems may compare their directional behavior and use the comparison to determine how to control one or both of the system. For example, if adjustments to both systems results in better performance, the systems may allow the adjustments. If one or both of the adjustments results in undesired behavior, the adjustments may be limited or reversed.

The first system may include a control system for monitoring and controlling the activities of the first system. The second system may include a control system for monitoring and controlling the operation of the second systems. At least one of the parameters monitored by the second system may be relevant to the operation of the first system. The parameter may not be monitored by the first system. Communication between the two systems may provide the first system with access to the data collected by the second system. The first system may use the data to effect the operational bounding of the first system's operation.

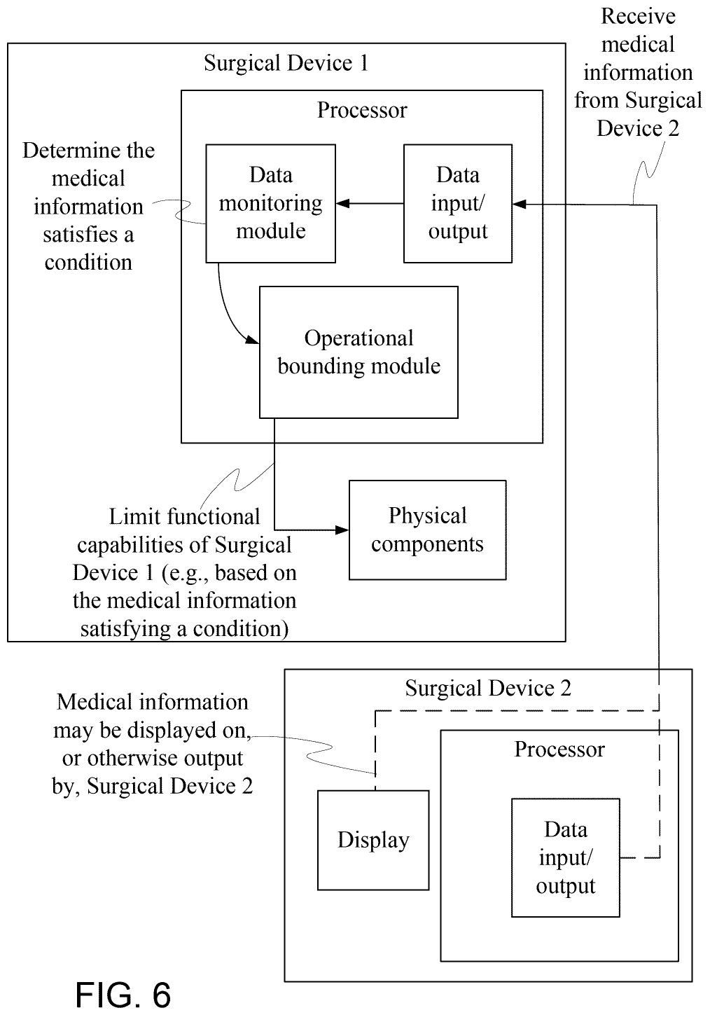

FIG. 6 illustrates an example of a first surgical device (e.g., Surgical Device 1) limiting its functionality based on patient data received from a second surgical device (e.g., Surgical Device 2). The second surgical device may include a processor with a data input/output module. The second surgical device may include a display, which may optionally display medical information. The first surgical device may determine that it is capable of obtaining medical information from a second surgical device. The first surgical device may obtain (e.g., receive) the medical information from the second surgical device. For example, the first surgical device may read the medical information from the display of the second surgical device. In another example, the second surgical device may output the medical information directly to a data input/output module of the first surgical device.

The first surgical device may include a processor with a data monitoring module and/or operational bounding module, as shown. The data monitoring module may receive the medical information and determine that the medical information satisfies a condition (e.g., patient temperature too low and/or the like). For example, determining that the medical information satisfies a condition may involve determining that operation of the first surgical device and operation of the second surgical device have created a negative feedback loop that negatively impacts patient health. The processor may indicate for the operational bounding module to limit the functional capabilities of the first surgical device (e.g., based on the medical information satisfying the condition). The operational bounding module may control physical components of the first surgical device to limit the functionality of the first surgical device (e.g., until the medical information is within an acceptable range again).

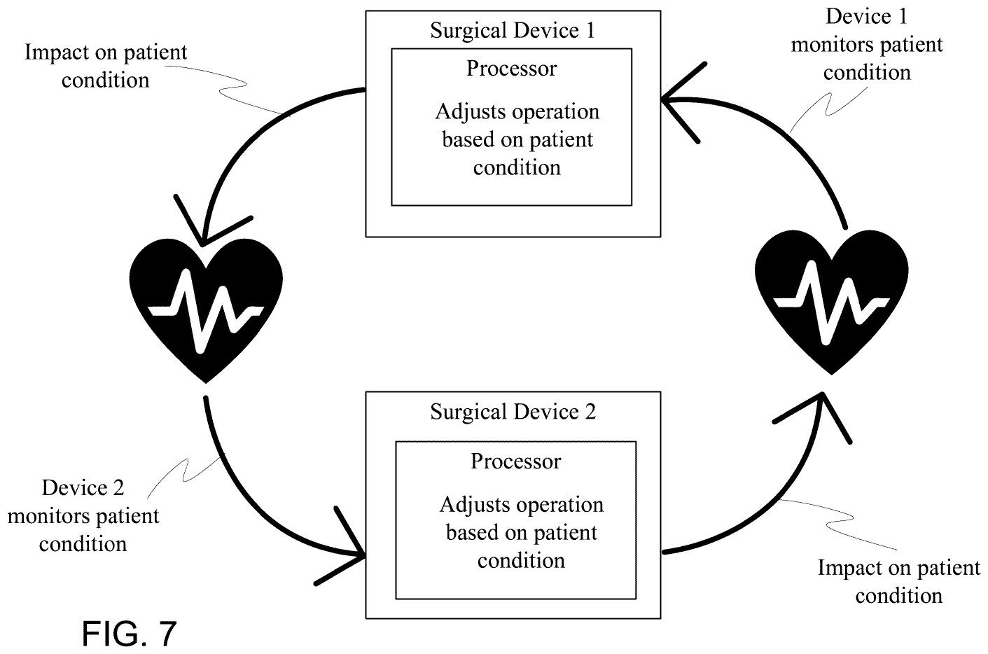

As illustrated in FIG. 7, first system may curtail full operational capabilities based on information monitored and provided by a second system. For example, the system may limit functional capabilities of the first surgical device such that the first surgical device operates in a manner, different than the first surgical device would operate without access to the medical information, so as to curtail negative impacts on a patient due to a negative feedback loop caused by normal operation of the first and second surgical devices. The second system may operate within the same area as the first system. The second system may provide the first system with information relating to the first system's behavior. The first system may change its operations based on a counter response from the second system.

Two separate smart systems may have a normal operational bounding (e.g., upper and lower limits). The systems may have an understanding that their control loops are interrelated in impacting the patient. In some circumstances (e.g., patient biomarkers are outside of the desired operational zone for one or both devices), the systems may make exemptions for one of their bounded control sets so that the systems may operate in concert to bring the patient parameters back within acceptable limits. The exemptions may be based on a directional improvement of the patient parameters. For example, reinforced control loop adjustment (e.g., where the closed loop monitor on one systems effects the monitored closed loop parameter of the other system) may be used. While the undesired self-reinforcing loop may be undesirable at times, the loop may be tolerated as long as the patient's biomarkers are moving in the correct direction and have not exceeded a maximum steady state magnitude. Once the discrepancy has been corrected, the normal operation functionalities and interactions may be resumed.

In an example, the first surgical device may be a ventilator. The device may determine that the medical information satisfies a condition by determining that carbon dioxide levels in a patient are above a threshold. The device may limit its functional capabilities by maintaining or increasing a tidal volume of the ventilator.

For example, oxygen (O₂) and carbon dioxide (CO₂) levels may be balanced. The O₂ and CO₂ levels may be monitored in the patient skin or blood flow. The ventilation rate may be controlled through the balance of volumetric airflow, O₂ and CO₂ concentrations within the airflow. Using a minimum volumetric tidal flow and minimum added oxygen may cause a buildup of CO₂ in the body (e.g., in procedures with long durations). This CO₂ buildup may inhibit healing short-term, change the pH of the blood & body, and/or limit recovery when the patient off the respirator based on standard volume breathing of the patient.

In an example, a surgeon may set the ventilator to a minimum tidal volume (e.g., to minimize the possibility of lung damage during inhalation). The O₂ may be controlled (e.g., open looped) by the surgeon at the beginning of the surgery. If the O₂ were controlled by oxygen partial pressure (PaO₂) patient monitoring, the ventilator may compensate to a lower tidal volume when the O₂ concentration is higher. However, this would increase the CO₂ build up (e.g., which is entirely tidal volume and rate based).

Smart PaO₂, CO₂, and heart rate monitoring may allow the ventilator respiration rate/volume and the O₂ concentration to be used. In this case, the ventilator rate may be the leading control and the O₂ may be a trailing control relative to the respiration rate (e.g., to reduce CO₂ and sustain O₂ blood gas levels).

The drugs delivered to the patient may impact the patient's core body temperature and/or metabolism. Based on the temperature or metabolic changes, the O₂ and/or CO₂ absorption rates may be impacted. Drug absorption may be impacted based on the patient's metabolic rate. At lower temperatures, the body may use less O₂. In this case, the negative effects of CO₂ may be less impactful. At lower temperatures, the patient's metabolic rate may decrease (e.g., thereby delaying drug absorption).

Tidal volumes may be 80 to 25 ml/kg between inhalation and exhalation, respectively. As a patient is put on a ventilator, the surgeon may set the ventilator to the lowest level (e.g., to avoid inadvertent injury). The O₂ volume may be set to the minimum to maintain the PaO₂ level. The minimum level tidal volume may not pull as much CO₂ out as O₂ going in. In this case, the CO₂ may cause acid to build up, lowering the pH of the patient (e.g., acidosis occurs at 7.35 and lower). A CO₂ limit may be used to increase tidal volume and lower O₂ to balance the CO₂ outlet with the O₂ inlet. The limit may continue to make tidal changes (e.g., based on time or CO₂ equilibrium levels) until the CO₂ and the O₂ are at target levels.

Too much CO₂ in the blood may be a sign of many conditions (e.g., lung diseases, Cushing's syndrome, kidney failure, metabolic alkalosis, in which the blood is not acidic enough, etc.). Acidosis may be caused by a buildup of CO₂ within the body. Acute kidney failure may be caused by a patient having acidosis for a relatively short amount of time (e.g., as little as ½ hour).

Normal acceptable levels of PaO₂ may be 75 to 100 millimeters of mercury (mm Hg), or 10.5 to 13.5 kilopascal (kPa). Normal acceptable levels of partial pressure of carbon dioxide (PaCO₂) may be 38 to 42 mm Hg (5.1 to 5.6 kPa). Normal acceptable levels of arterial blood pH may be 7.38 to 7.42. Normal acceptable levels of oxygen saturation (SaO₂) may be 94% to 100%. Normal acceptable levels of bicarbonate (HCO₃) may be 22 to 28 milliequivalents per liter (mEq/L).

The leading control variable relationship may be reversed in some examples.

With O₂ introduction and long-term supplementation, the body may (e.g., normally) decrease tidal volume (e.g., naturally due to the body being keyed to O₂, not CO₂). This may amplify the desirability for the externally-measured systems to monitor CO₂ (e.g., on which the body does not have a closed loop control).

Feature(s) associated with controlled ventilation and respiratory drive are provided herein. O₂ and CO₂ may be balanced in mechanical ventilation.

Mechanical hyperventilation may deplete (e.g., rapidly and abnormally deplete) CO₂ tissue reserves and blood bicarbonate. This may undermine respiratory drive for hours (e.g., until metabolic activity can replenish CO₂ levels). Hyperventilated patients may breathe and oxygenate effectively. The respiratory drive of hyperventilated patients may depend on their conscious awareness of surgical pain and psychological stimulation that provides an artificial stimulus to breathe. During this vulnerable period, even very small doses of opioids may unexpectedly obliterate the sole remaining source of respiratory drive. In this case, seemingly awake, alert, and fully recovered patients may unpredictably stop breathing.

In an example, the first surgical device may be a ventilator. The device may determine that the medical information satisfies a condition by determining that carbon dioxide levels in a patient are below a threshold (e.g., that the patient is hyperventilated). The device may limit its functional capabilities by decreasing a tidal volume of the ventilator.

Ventilation rate and volume may be controlled. For example, a medical professional may perform a controlled lung collapse. The lung collapse may be controlled using thoracic cavity internal pressure.

Insufflation pressure, suction magnitude, and smoke evacuation magnitude may be controlled to maintain a functional surgical working space. In this example, one of the systems may cause the pressure imbalance that another system is trying to overcome. The first surgical device may be a smoke evacuation device. The device may determine that the medical information satisfies a condition by determining that a patient core body temperature is below a threshold. The device may limit functional capabilities of the first surgical device by reducing the rate of smoke evacuation.

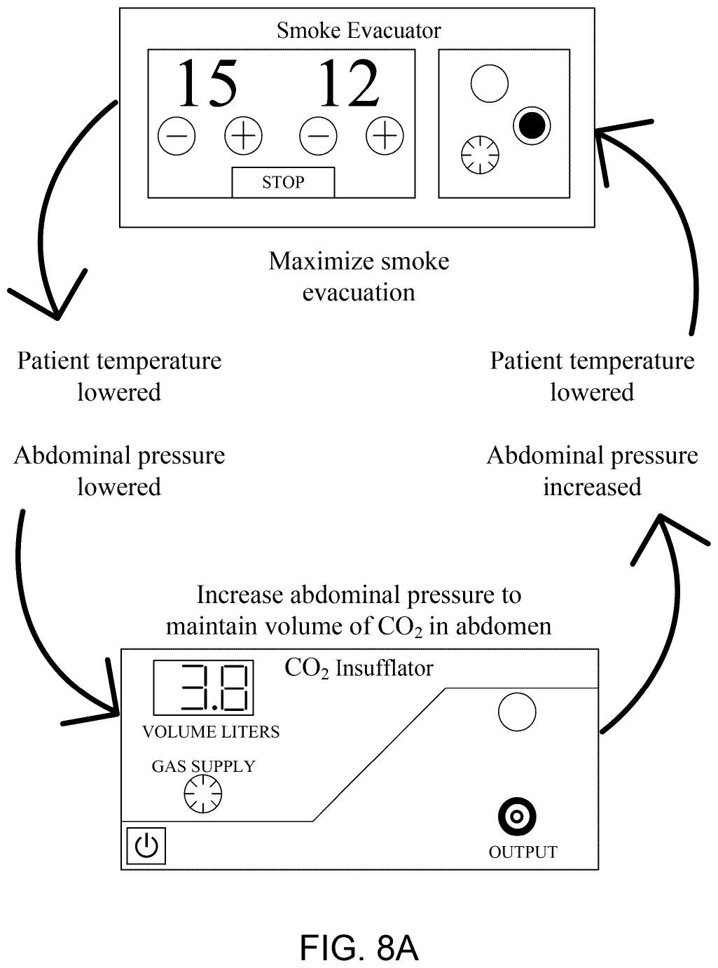

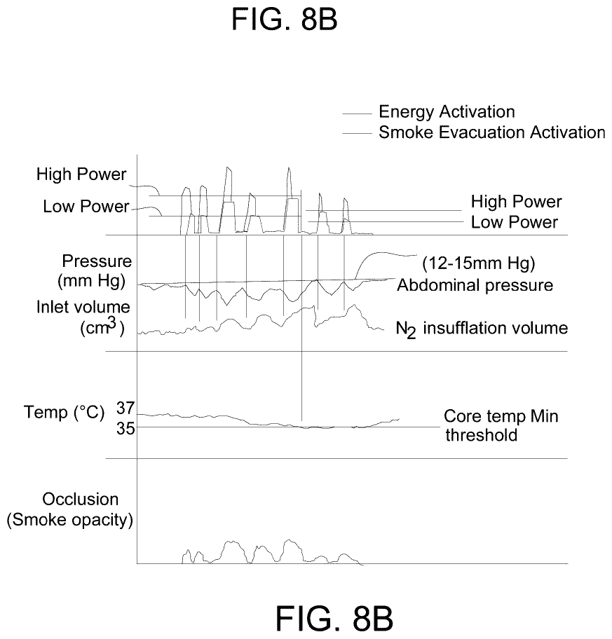

For example, as illustrated in FIG. 8A, as the use of suction or smoke evacuation is increased, the rate of cold CO₂ insufflation may increase (e.g., thereby causing a hypothermic response). The abdominal pressure to main the working space may use more CO₂ as gas is vented and suctioned away. The patient monitoring may be used to regulate the system of the thermal instability. For example, the patient monitoring system may request that the smoke evacuator be used less or slower. In this case, the smoke evacuator may ask the advanced energy generator to use a lower power (e.g., thereby causing less smoke rather than causing the hypothermic response).

The smoke evacuation may remove the smoke to improve visibility and reduce abdominal pressure. The pressure drop may increase the input volume of cold gas, which in turn causes the patient's core temperature to drop. The core temperature loss may cause hypothermia. The hypothermic response may cause the surgeon to lower the smoke evacuation rate and/or the energy power level of the energy generator. FIG. 8B is a graph that illustrates the effect of these devices on the patient's core temperature.

Enforcing a boundary limit of a following device may cause a reverse in the closed loop control (e.g., back to the cause of the adjustment).

Patient core body temperature may be controlled through interactive environmental, localized patient heating and cooling, and incidental heat loss (e.g., using insufflation and/or suction magnitudes). In an example, the first surgical device may be a patient heating system. The device may determine that the medical information satisfies a condition by determining that a patient core body temperature or a patient extremity body temperature is below a threshold. The device may limit functional capabilities of the first surgical device by increasing patient heating.

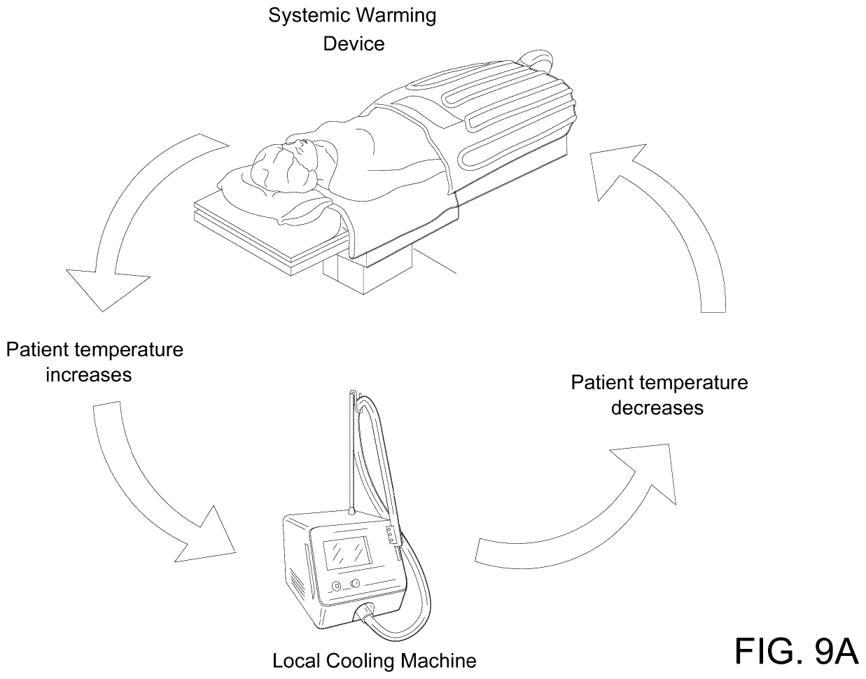

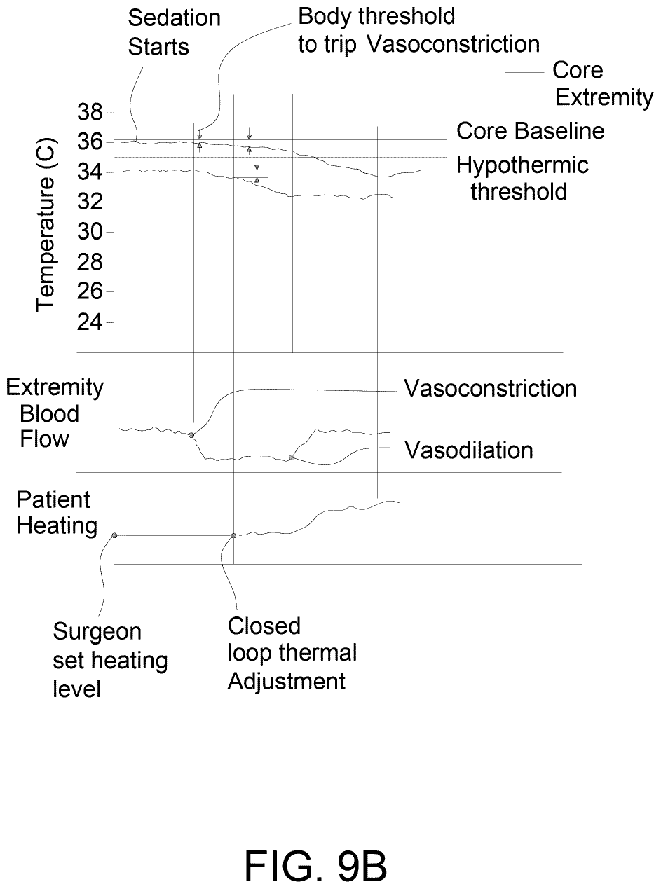

As illustrated in FIGS. 9A and 9B, thermo-regulation may be used in response to the body's vasodilatation and/or vasoconstriction based on the patient core temperature deviation.

Sedation may cause the patient to lose core body temperature (1-2° C.), which may cause hypothermia (e.g., at around 35° C.). To compensate for this loss, a surgeon may use a patient heating device, which may be (pre)set to compensate for the cold room temperature and the impacts of sedation (e.g., a bear-hugger heating vest may be used to counteract the heat loss). The surgeon may set the thermo load input (e.g., at the beginning of the surgery) to compensate for the sedation loss. In some cases (e.g., colorectal surgery), local cooling may be used to prevent ischemia (e.g., of the colon) due to interruption of blood flow. The combination of sedation cooling and local cooling may (e.g., initially) result in vasoconstriction (e.g., as the body tried to maintain core temperature with the aid of the systemic patient warming). Specifically, the body may vasoconstrict the blood flow to extremities.

As the procedure continues, the local cooling may have a more global effect on the body. If the patient's core temperature drops more than 2° C., the body may reverse the vasoconstriction to a vasodilation state. The vasodilation opens the flow of cold blood to the extremities, which may rapidly increase the core temperature loss. The patient heating system may operate in an open loop manner (e.g., set by the surgeon) or a closed loop manner in which the heating may change by request or after the body temperature falls below a threshold or the change in temperature exceeds a limit. If the patient heating system lags too much, the rapid re-heating may cause additional cold blood flow to the heart. This may cause arrhythmias and potentially heart failure.

It may be beneficial, therefore, for the heating system to monitor the extremity temperature or blood flow as a means to preemptively determine an appropriate heating rate (e.g., so that the heating system does not fall too far behind, causing the rapid heating issue described above). ΔT_{c} may refer to the drop in core temperature. ΔTₑ may refer to the drop in the temperature of the extremities. Either may be used as an open-closed loop control of the heating

Metabolic uptake of medicine may be based on thermal levels of the patient core temperature. As the core temperature is reduced, the medication dosage may be (e.g., automatically) adjusted based on the lower metabolic uptake. If the body then reheats itself, the dosing and the existing levels of medicine have to be reduced before the metabolic uptake increases too much.

For closed loop control of the local cooling and the patient heating, the patient core temperature and extremity temperatures may be monitored. If the system detects the vasodilation trigger, the system may stop increasing local cooling and/or increase the patient systemic heating to prevent the excessive rapid loss of heat (e.g., which would trigger a secondary rapid heating response).

Physiological compensation may cause the system to reverse its closed loop control adjustments. For example, in the case of heart surgery, the surgeon may (e.g., intentionally) put the patient into a therapeutic hypothermic state (e.g., to prevent ischemic damage during the intervention in the blood supply to the heart). malignant airway obstruction (MAO) deterministic monitoring may be implemented. The most reliable measure of endotracheal intubation may be with direct visualization (e.g., during laryngoscopy) and the presence of persistently elevated end-tidal carbon dioxide (ETCO₂).

Positive return of end-tidal carbon dioxide alone may or may not confirm endotracheal placement of the endotracheal tube (ETT). Greater than 30 mm Hg ETCO₂ being sustained for three breaths minimum may be used to confirm tracheal placement. If a patient drank a bicarbonate solution or carbonated beverage before intubation, this also alter the measured ETCO₂. Capnography may use cardiac output and the gas exhaust, which may, for example, be used to quantify CO₂ outlet.

In an example, the viable operational envelope of a system or device may be limited based on the possibility of two moving smart systems occupying the common space at the same time to avoid collisions. For example, the first surgical device may be a first robotic arm in control of a first surgical instrument and the second surgical device may be a second robotic arm in control of a second surgical instrument. The first surgical device may determine that the medical information satisfies a condition by determining that a current movement trajectory of the first surgical device will cause a collision between the first surgical instrument and the second surgical instrument. In response, the first surgical device may limit functional capabilities of the first surgical device by stopping or changing the trajectory of the first surgical device.

An adaptive robot-to-robot no-fly zone based on an aspect of a first robot arm (e.g., location of the second robot cart, its robot arm position, movements, required operational envelope, etc.) may be used to limit the space that a second robot arm (e.g., from either the same robot or a separate robot) is allowed to use. The no-fly zone may be inside or outside of the patient.

For example, a first laparoscopic multi-cart robot may be used to for dissection and resection of a mid-parenchyma tumor that is on the junction of two segments. The surgeon may attempt to separate out the tumor from the artery and vein (e.g., to avoid removing two full segments). The surgeon may realize during surgery that the tumor has invaded the bronchus. To determine penetration depth and the extent of invasion, the surgeon may bring in a flexible endoscope controlled with a separate robot. The introduction of the second robot may not involve repositioning of the existing first robot cart. One of the carts may be positioned towards the head of the patient and have a working envelope outside of the body that encompasses some of the space now occupied by the flexible endoscopy robot and its operating envelope.

The second robot may establish communication with the first robot. The second robot may identify a its location and size dimensions. The second robot may define a minimum space in which it intends to operate. The second robot may inform the first robot of the reduced operational envelope in which the first robot will be able to operate without entangling the robots. This regulation of the first robot by the second robot may involve defining a space reduction and actively monitoring the first robot arm. The restriction may involve defining a portion of the full operating envelope in which the first robot may no longer operate. The restriction may involve actively adjusting regulation of the space (e.g., that changes as the first robot coordinates its operation with the flexible endoscopy robot). In this case, the space may be reduced only as needed to perform certain actions, while allowing the first robot to occupy the shared space (e.g., if the second robot does not need that space). If both robots intend to occupy the same shared space, the robots may negotiate based on priority, user input, or computational ordering that would allow the motions to be choreographed. The choreographs motion may allow the robots to move around each other through a series of pre-calculated alternating motions (e.g., to allow them to move around each other without adverse interactions).

As the flexible endoscopy robot is brought into the OR and setup, the user may input the location and operational window for the endoscopy robot, or one of the smart systems may define the location and operational envelopes of both robots (e.g., relative to each other).

A hub and a room-based camera may be used to identify the exact location, shape, and/or operational window of the devices (e.g., based on the setup of the devices). For example, multiple perspective cameras with overlapping image coverage may be used. As another example, fewer cameras and laser alignment and/or light direction and ranging (Lidar) may be used as a means to detect distances and even structured light as a means to define shapes and volumes.

Subtractive operational envelope reduction may be used to reduce the space used by a smart system. For example, one of the smart systems may be capable of using a space, but may no longer be allowed to use that space based on a use or priority of another system with regard to the shared envelope.

The first smart device's position, motions, articulation, energy potential, and/or activation state may cause an adjustment of an adjacent device in near proximity. For example, a conductive end-effector's zone of occlusion and interaction may be modified depending on whether or not one of the end-effectors monopolar energy is active or active and above a certain threshold. This may prevent inadvertent energizing of the non-monopolar device based on either inadvertent contact or close proximity in contact with the same tissue (e.g., where the second device could become part of the return path). The coordination may minimize inadvertent burns to the patient away from the surgical site based on the continuity path of the second device's other tissue contacts.

The operational window of a first device may be adjusted due to the proximity of another device to limit the proximity of the second device (e.g., if the first is active). The first device may be prevented from activating based on proximity of the second device (e.g., in order to reduce the risk of interference between devices). For example: the activation of energizing a first device may be limited if the first device is in close proximity to a second device that has sensing means that would be effected by using the energy nearby.

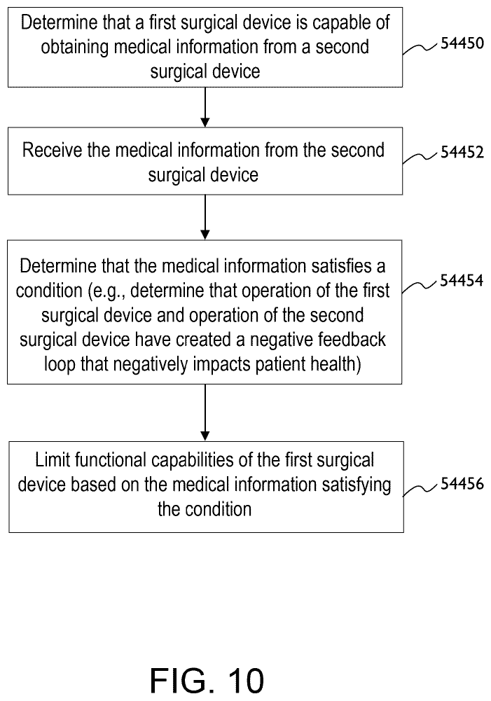

FIG. 10 illustrates an example method that may be performed by a surgical device. As shown at 54450, the method may involve determining that the first surgical device is capable of obtaining medical information from a second surgical device. The method may involve, at 54452, receiving the medical information from the second surgical device. The method may involve, at 54454, determining that the medical information satisfies a condition. Determining that the medical information satisfies a condition may involve determining that operation of the first surgical device and operation of the second surgical device have created a negative feedback loop that negatively impacts patient health. The method may involve, at 54456, limiting functional capabilities of the first surgical device based on the medical information satisfying the condition.

What is claimed:

1. A first surgical device comprising a processor configured to:
determine that the first surgical device is capable of obtaining medical information from a second surgical device;
receive the medical information from the second surgical device;
determine that the medical information satisfies a condition, wherein the processor being configured to determine that the medical information satisfies a condition comprises the processor being configured to determine that operation of the first surgical device and operation of the second surgical device have created a negative feedback loop that negatively impacts patient health; and
limit functional capabilities of the first surgical device based on the medical information satisfying the condition.

2. The first surgical device of claim 1, wherein the processor being configured to limit functional capabilities of the first surgical device based on the medical information satisfying the condition comprises the processor being configured to operate in a manner, different than the first surgical device would operate without access to the medical information, so as to curtail negative impacts on a patient due to a negative feedback loop caused by normal operation of the first and second surgical devices.

3. The first surgical device of claim 1, wherein:
the first surgical device is a smoke evacuation device;
the processor being configured to determine that the medical information satisfies a condition comprises the processor being configured to determine that a patient core body temperature is below a threshold; and
the processor being configured to limit functional capabilities of the first surgical device comprises the processor being configured to reduce the rate of smoke evacuation.

4. The first surgical device of claim 1, wherein:
the first surgical device is a patient heating system;
the processor being configured to determine that the medical information satisfies a condition comprises the processor being configured to determine that a patient core body temperature or a patient extremity body temperature is below a threshold; and
the processor being configured to limit functional capabilities of the first surgical device comprises the processor being configured to adjust patient heating.

5. The first surgical device of claim 1, wherein:
the first surgical device is a ventilator;
the processor being configured to determine that the medical information satisfies a condition comprises the processor being configured to determine that carbon dioxide levels in a patient are above a threshold; and
the processor being configured to limit functional capabilities of the first surgical device comprises the processor being configured to adjust a tidal volume of the ventilator.

6. The first surgical device of claim 1, wherein:
the first surgical device is a ventilator;
the processor being configured to determine that the medical information satisfies a condition comprises the processor being configured to determine that carbon dioxide levels in a patient are below a threshold; and
the processor being configured to limit functional capabilities of the first surgical device comprises the processor being configured to decrease a tidal volume of the ventilator.

7. The first surgical device of claim 1, wherein:
the first surgical device is a first robotic arm in control of a first surgical instrument;
the second surgical device is a second robotic arm in control of a second surgical instrument;
the processor being configured to determine that the medical information satisfies a condition comprises the processor being configured to determine that a current movement trajectory of the first surgical device will cause a collision between the first surgical instrument and the second surgical instrument; and
the processor being configured to limit functional capabilities of the first surgical device comprises the processor being configured to stop or change the trajectory of the first surgical device in response to determining that the current movement trajectory of the first surgical device will cause the collision.

8. A method performed by a first surgical device, the method comprising:
determining that the first surgical device is capable of obtaining medical information from a second surgical device;
receiving the medical information from the second surgical device;
determining that the medical information satisfies a condition, wherein determining that the medical information satisfies a condition comprises determining that operation of the first surgical device and operation of the second surgical device have created a negative feedback loop that negatively impacts patient health; and
limiting functional capabilities of the first surgical device based on the medical information satisfying the condition.

9. The method of claim 8, wherein limiting functional capabilities of the first surgical device based on the medical information satisfying the condition comprises operating in a manner, different than the first surgical device would operate without access to the medical information, so as to curtail negative impacts on a patient due to a negative feedback loop caused by normal operation of the first and second surgical devices.

10. The method of claim 8, wherein:
the first surgical device is a smoke evacuation device;
determining that the medical information satisfies a condition comprises determining that a patient core body temperature is below a threshold; and
limiting functional capabilities of the first surgical device comprises reducing the rate of smoke evacuation.

11. The method of claim 8, wherein:
the first surgical device is a patient heating system;
determining that the medical information satisfies a condition comprises determining that a patient core body temperature or a patient extremity body temperature is below a threshold; and
limiting functional capabilities of the first surgical device comprises adjusting patient heating.

12. The method claim 8, wherein:
the first surgical device is a ventilator;
determining that the medical information satisfies a condition comprises determining that carbon dioxide levels in a patient are above a threshold; and
limiting functional capabilities of the first surgical device comprises adjusting a tidal volume of the ventilator.

13. The method of claim 8, wherein:
the first surgical device is a ventilator;
determining that the medical information satisfies a condition comprises determining that carbon dioxide levels in a patient are below a threshold; and
limiting functional capabilities of the first surgical device comprises decreasing a tidal volume of the ventilator.

14. The method of claim 8, wherein:
the first surgical device is a first robotic arm in control of a first surgical instrument;
the second surgical device is a second robotic arm in control of a second surgical instrument;
determining that the medical information satisfies a condition comprises determining that a current movement trajectory of the first surgical device will cause a collision between the first surgical instrument and the second surgical instrument; and
limiting functional capabilities of the first surgical device comprises stopping or changing the trajectory of the first surgical device in response to determining that the current movement trajectory of the first surgical device will cause the collision.
